SEQUENCE LISTING (3 sequences)

SEQ ID NO: 1 - protein, 6 aa, Artificial Sequence
  Apoptotic cell-targeting peptide, ApoPep-1
CQRPPR

SEQ ID NO: 2 - DNA, 20 bp, Artificial Sequence
  Forward primer
agcggaccagattatcgcta

SEQ ID NO: 3 - DNA, 20 bp, Artificial Sequence
  Reverse primer
aacccctcaagacccgttta

(19) United States
(12) Patent Application Publication
(10) Pub. No.: US 2012/0258038 A1
(43) Pub. Date: Oct. 11, 2012

(54) USES OF APOPTOTIC CELL-TARGETING PEPTIDES, LABEL SUBSTANCES AND LIPOSOMES CONTAINING A THERAPEUTIC AGENT FOR PREVENTING, TREATING OR THERAPEUTICALLY DIAGNOSING APOPTOSIS-RELATED DISEASES

(75) Inventors: Byung-Heon Lee, Daegu (KR); In San Kim, Daegu (KR); Yu Kyoung Oh, Seoul (KR)

(73) Assignee: Kyungpook National University Industry-Academic Cooperation Foundation, Daegu (KR)

(21) Appl. No.: 13/470,076

(22) Filed: May 11, 2012

Related U.S. Application Data
(63) Continuation of application No. PCT/KR10/07999, filed on Nov. 12, 2010.

(30) Foreign Application Priority Data
Nov. 13, 2009 (KR) 10-2009-0109955

(51) Int. Cl. A61K 38/08; A61K 49/00; A61P 35/00; A61P 9/10; A61K 9/127; A61K 51/12

(52) U.S. Cl. 424/1.11; 424/450; 514/1.1; 424/9.6; 424/9.1

(57) ABSTRACT

The present invention relates to a composition for preventing, treating or theranosis of apoptosis-related diseases comprising liposome comprising apoptotic cell-targeting peptides, label substances and a therapeutic agent. The present invention may be used for drug delivery to the apoptotic cells in cancer or tumor mass, the apoptotic myocardial cells in myocardial infarction lesion, the apoptotic stroke cells in stroke lesion, the apoptotic cells in arteriosclerosis lesion and further may be used for detection of the cells and imaging diagnosis. Therefore, it can be used for theranosis as well as preventing or treating the diseases.

### CROSS-REFERENCE TO RELATED APPLICATIONS

This is a continuation of International Application No. PCT/KR2010/007999 filed on Nov. 12, 2010, which claims the priority to Korean Application No. 10-2009-0109955 filed on Nov. 13, 2009, which applications are incorporated herein be reference.

### TECHNICAL FIELD

The present invention relates to uses for preventing, treating or theranosis of apoptosis-related diseases of liposome comprising apoptotic cell-targeting peptides, label substances and a therapeutic agent. More particularly, it relates to uses of liposome for preventing, treating or theranosis of an apoptosis-related disease, such as cancer, myocardial infarction, stroke, arteriosclerosis, or the like, which is labeled with an apoptotic cell-targeting peptide (amino acid sequence CQRPPR, ApoPep-1) and a labeling material on the surface thereof, and contains a therapeutic agent.

### BACKGROUND ART

Apoptosis indicates a phenomenon resulting in the death of unnecessary cells or dangerous cells themselves, which is for life conservation of an individual. In Greek, apoptosis means “to fall.” It describes the falling of cell organisms, and was named by comparing the process of cell death to the falling of petals from a flower, which was first observed in 1972 by Kerr, et al (Kerr et al., Br J Cancer, 1972, 26:239-257). Apoptosis plays an important role in physiological events, including cell development, cell differentiation, cellular immunity and the like (Meier et al., Nature, 2000, 407:796-801). Meanwhile, apoptosis is important in several pathological conditions and diseases. For example, successful treatment with antitumor agents involves much apoptosis in the tumor tissue (Thomson, Science, 1995, 267:1456-1462). On the other hand, decreased apoptosis results in formation of tumors.

Apoptosis is very important in clinical diagnosis and treatment. In other words, imaging of apoptosis may be of great help in monitoring of the cancer therapeutic effect following antitumor agent treatment. Also, a selective delivery of a therapeutic agent or a cytoprotective medicine to an active apoptosis site may significantly improve the therapeutic effect while reducing side effects.

One of the early events occurring in apoptotic cells is the change of the distribution of phospholipids that constitute the cell membrane. The most characteristic thing among them is the exposure of phosphatidylserine to the outside of the cell membrane. Normally, phosphatidylserine is kept inside the cell membrane, but when a cell receives an apoptotic signal or when a red blood cell is aged, it is exposed to the outside of the cell membrane (Fadeel, B. et al., Cell Mol Life Sci, 2003, 60:2575-2585). A macrophage recognizes the exposed phosphatidylserine through a receptor on the cell surface and phagocytoses the apoptotic cell (Fadok, V. A. et al., J immunol 1992, 148:2207-2216; Fadok, V. A. et al., Nature 2000, 405:85-90; Park, S. Y. et. al., Cell Death Differ, 2008, 15:192-201). Especially, a large number of tumor cells show an increase of expression of phosphatidylserine outside the cell membrane (Utsugi, T. et al., Cancer Res. 1991, 15:3062-3066; Ran, S. et al., Cancer Res. 2002, 62:6132-6140; Woehlecke, H. et al., Biochem J. 2003, 376:489-495). Also, the vascular endothelial cells of a small vessel in a tumor tissue expose phosphatidylserine outside of the cell membrane (Ran, S. et al., Cancer Res. 2002, 62:6132-6140; Zwaal, R. F. A. et al., Blood. 1997, 89:1121-1132). Accordingly, due to such roles of phosphatidylserine, in various situations especially including tumors, the phosphatidylserine is deemed as a target substance for diagnosis, treatment, and treatment monitoring.

At present, the protein annexin V is generally used to detect phosphatidylserine on the surface of apoptotic cells. It is a protein having a molecular weight of 36 kDa, and binds to phosphatidylserine with strong affinity (Vermes, I. et al., Immunol Methods. 1995, 184:39-51). Meanwhile, although annexin V is a very useful target substance or probe for in vitro application, its in vivo application is reported to be restricted because of, for example, slow removal out of the body due to its large molecular weight (Vermeersch, H., et al., Nucl Med Commun. 2004, 25:259-263; Belhocine, T. Z. et al., J Proteome Res. 2004, 3:345-349).

Meanwhile, a liposome is a spherical vesicle composed of a phospholipid bilayer surrounding an aqueous phase. A lipid membrane is composed of amphipathic phospholipids including two hydrophobic fatty acid groups and a hydrophilic phosphate group. The phospholipids form bilayers in an aqueous solution, and may form closed vesicles like artificial cells. In the bilayer structure, non-polar fatty acid tails exist toward the interior of the bilayer while polar heads exist toward the exterior of the bilayer. A liposome is largely classified into two kinds of liposomes according to the number of lamellars. A single-lamellar liposome includes one lipid bilayer. A multi-lamellar liposome has two or more lipid bilayers. Liposomes may be produced by various methods (Cullis et al., in: Liposomes, From Biophysics to Therapeutics (M. J. Ostro, ed.), Marcel Dekker, pp. 39-72 (1987).

The entrapping of a drug in liposomes decreases the toxicity of the drug, and increases the effect of the drug while enhancing the therapy. Also, liposomes, like other specific substances in a circulatory system, may be generally entrapped by phagocytes of a reticuloendothelial cell system in a tissue having an oval capillary vessel, and then directly transferred to an intracellular infected site.

Theranosis (theragnosis, theragnostics) is a compound word of therapy with diagnosis (diagnostics), which indicates a therapy technique combined with a diagnosis technique. In such a case, a response in a therapeutic agent for each patient may be determined and applied to selection of a therapeutic method. This may prevent misuse or abuse of drugs, and highly distribute to improvement of a therapeutic effect. (Frederic P et al., Crit. Care Med, 2009, Vol. 37, No. 1(Suppl.) S50-S58; Haglund E et al. Annals of Biomedical Engineering, Vol. 37, No. 10, 2009, pp. 2048.2063; Ozdemir V et al., Nature Biotechnology, Vol. 24, No. 8, 2006, 942-946)

### Summary of the Disclosure

Accordingly, the present inventors have worked to develop novel proteins or fragments thereof capable of specifically and early targeting apoptotic cells in vivo. As a result, they have developed a peptide having an amino acid sequence of CQRPPR, and named it ApoPep-1. Also, they verified that a therapeutic agent-containing liposome labeled with the peptide shows a higher therapeutic effect than a liposome not labeled with the peptide, and also a labeling material as well as the peptide can be labeled for use in theranosis. Based on this finding, they have completed this invention.

Accordingly, an object of the present invention is to provide liposome comprising apoptotic cell-targeting peptides, label substances and a therapeutic agent including an anticancer agent and use thereof.

To achieve the above object, the present invention provides a drug delivery composition comprising liposome comprising apoptotic cell-targeting peptides and therapeutic agents as an active ingredient.

To achieve another object, the present invention provides a composition for preventing and treating cancer comprising liposome comprising apoptotic cell-targeting peptides and an anticancer agent as an active ingredient.

To achieve still another object, the present invention provides a composition for theranosis of cancer comprising liposome comprising apoptotic cell-targeting peptides, label substances and an anticancer agent as an active ingredient.

To achieve still another object, the present invention provides a composition for preventing and treating stroke comprising liposome comprising apoptotic cell-targeting peptides and therapeutic agents for stroke as an active ingredient.

To achieve still another object, the present invention provides a composition for theranosis of stroke comprising liposome comprising apoptotic cell-targeting peptides, label substances and therapeutic agents for stroke as an active ingredient.

To achieve still another object, the present invention provides a composition for preventing and treating myocardial infarction comprising liposome comprising apoptotic cell-targeting peptides and therapeutic agents for myocardial infarction as an active ingredient.

To achieve still another object, the present invention provides a composition for theranosis of myocardial infarction comprising liposome comprising apoptotic cell-targeting peptides, label substances and therapeutic agents for myocardial infarction as an active ingredient.

To achieve still another object, the present invention provides a composition for preventing and treating arteriosclerosis comprising liposome comprising apoptotic cell-targeting peptides and therapeutic agents for arteriosclerosis as an active ingredient.

To achieve still another object, the present invention provides a composition for theranosis of arteriosclerosis comprising liposome comprising apoptotic cell-targeting peptides, label substances and therapeutic agents for arteriosclerosis as an active ingredient.

To achieve still another object, the present invention provides a method for drug delivery comprising the step of administering to a subject in need thereof an effective amount of liposome comprising apoptotic cell-targeting peptides and therapeutic agents.

To achieve still another object, the present invention provides use of liposome comprising apoptotic cell-targeting peptides and therapeutic agents for preparing an agent for drug delivery.

To achieve still another object, the present invention provides a method for preventing and treating cancer comprising the step of administering to a subject in need thereof an effective amount of liposome comprising apoptotic cell-targeting peptides and an anticancer agent.

To achieve still another object, the present invention provides use of liposome comprising apoptotic cell-targeting peptides and an anticancer agent for preparing an agent for preventing and treating cancer.

To achieve still another object, the present invention provides a method for theranosis of cancer comprising the step of administering to a subject in need thereof an effective amount of liposome comprising apoptotic cell-targeting peptides, label substances and an anticancer agent.

To achieve still another object, the present invention provides use of liposome comprising apoptotic cell-targeting peptides, label substances and an anticancer agent for preparing an agent for theranosis of cancer.

To achieve still another object, the present invention provides a method for preventing and treating stroke comprising the step of administering to a subject in need thereof an effective amount of liposome comprising apoptotic cell-targeting peptides and therapeutic agents for stroke.

To achieve still another object, the present invention provides use of liposome comprising apoptotic cell-targeting peptides and therapeutic agents for stroke for preparing a therapeutic agent for stroke.

To achieve still another object, the present invention provides a method for theranosis of stroke comprising the step of administering to a subject in need thereof an effective amount of liposome comprising apoptotic cell-targeting peptides, label substances and therapeutic agents for stroke.

To achieve still another object, the present invention provides a use of liposome comprising apoptotic cell-targeting peptides, label substances and therapeutic agents for stroke for preparing an agent for theranosis of stroke.

To achieve still another object, the present invention provides a method for preventing and treating myocardial infarction comprising the step of administering to a subject in need thereof an effective amount of liposome comprising apoptotic cell-targeting peptides and therapeutic agents for therapeutic agent for myocardial infarction.

To achieve still another object, the present invention provides use of liposome comprising apoptotic cell-targeting peptides and therapeutic agent for myocardial infarction for preparing an agent for preventing and treating myocardial infarction.

To achieve still another object, the present invention provides a method for theranosis of myocardial infarction comprising the step of administering to a subject in need thereof an effective amount of liposome comprising apoptotic cell-targeting peptides, label substances and therapeutic agent for myocardial infarction.

To achieve still another object, the present invention provides use of liposome comprising apoptotic cell-targeting peptides, label substances and therapeutic agent for myocardial infarction for preparing an agent or theranosis of myocardial infarction.

To achieve still another object, the present invention provides a method for preventing and treating arteriosclerosis comprising the step of administering to a subject in need thereof an effective amount of liposome comprising apoptotic cell-targeting peptides and therapeutic agent for arteriosclerosis.

To achieve still another object, the present invention provides use of liposome comprising apoptotic cell-targeting peptides and therapeutic agent for arteriosclerosis for preparing an agent for preventing and treating arteriosclerosis.

To achieve still another object, the present invention provides a method for theranosis of arteriosclerosis comprising the step of administering to a subject in need thereof an effective amount of liposome comprising apoptotic cell-targeting peptides, label substances and therapeutic agent for arteriosclerosis.

To achieve still another object, the present invention provides a use of liposome comprising apoptotic cell-targeting peptides, label substances and therapeutic agent for arteriosclerosis for preparing an agent for theranosis of arteriosclerosis.

### BRIEF DESCRIPTION OF DRAWINGS

FIG. 1 shows images obtained as follows. Apoptosis was induced in various kinds of cells (A549, H460, HeLa, L132, RAW) by treating with etoposide. Images were obtained for the fluorescence resulting from the binding with the inventive peptide (ApoPep-1) (B, F, J, N, R), annexin V staining of the cells (C, G, K, O, S), and merges thereof (D, H, L, P, T). (A, E, I, M, and Q) are merged images of the cells (as a control group) in which binding of the cells, not treated with an apoptosis-inducing drug, to the inventive peptide (ApoPep-1) or annexin V was imaged;

FIG. 2 shows images obtained as follows. Apoptosis was induced in A549 tumor cells by treating with etoposide. Images were obtained by staining with red fluorescence-labeled annexin V after pre-treating with annexin V without a fluorescence label at concentrations of 0 μM(A), 200 μM(B) and 1000 μM(C). Further, after pre-treating with annexin V at a concentration of 1000 μM, images were obtained for binding with the inventive peptide (ApoPep-1) (E), nuclear staining (D) and a merge thereof (F);

FIG. 3 shows FACS analysis results of binding of A549 tumor cells, treated with or without etoposide, to annexin V (A), the inventive peptide(ApoPep-1) or a control peptide (Control)(B). Herein, the abscissa represents the degree of binding to annexin V or the peptide, and the ordinate represents the degree of PI (propodim iodide) staining. (A549: etoposide non-treated group; Etoposide: etoposide treated group);

FIG. 4 schematically shows structures of a doxorubicin-containing liposome labeled with a Cy7.5 near infrared fluorescent reagent, and a doxorubicin-containing liposome labeled with both an apoptosis-targeting peptide(ApoPep-1) and a Cy7.5 near infrared fluorescent reagent;

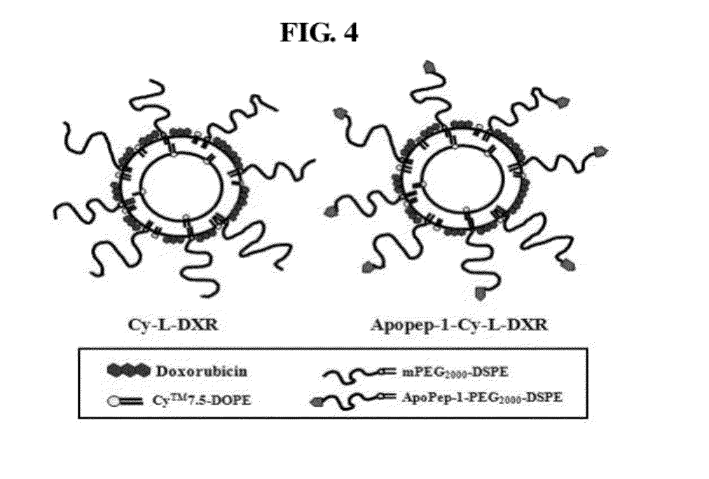

FIG. 5 shows the measurement results of size (A) and body weight (B) of a tumor after doxorubicin or doxorubicin-containing liposome, labeled or not labeled with an apoptosis-targeting peptide(ApoPep-1), was intravenously injected into an H460 tumor-xenotransplanted nude mouse for a total of seven times with a 2 day interval. Also, FIG. 5(C) shows the measurement result of a size of a tumor after the drug was injected into an A549 tumor-xenotransplanted nude mouse in the same manner as described in FIG. 5(A) (HEPES: buffer solution; DXR: doxorubicin; L-DXR: doxorubicin-containing liposome; ApoPep-1-L-DXR: ApoPep-1-labeled doxorubicin-containing liposome);

FIG. 6 schematically shows an experimental plan for intravenously injecting a liposome into a tumor-xenotransplanted mouse so as to carry out theranosis, that is, both diagnosis and treatment (HEPES: buffer solution; DXR: doxorubicin; L-DXR: doxorubicin-containing liposome; ApoPep-1-L-DXR: ApoPep-1-labeled doxorubicin-containing liposome; Cy-L-DXR: Cy7.5 near infrared fluorescent reagent-labeled doxorubicin-containing liposome; ApoPep-1-L-DXR: ApoPep-1 and near infrared fluorescent reagent-labeled doxorubicin-containing liposome). Each arrow indicates a point of time when each liposome was injected;

FIG. 7 shows images obtained as follows. For theranosis, that is, both diagnosis and treatment, near infrared fluorescence images of a tumor site were obtained after a doxorubicin-containing liposome was intravenously injected into a tumor-xenotransplanted nude mouse, labeled with Cy7.5 together with an apoptosis-targeting peptide(ApoPep-1), with a 2 day interval for a total of once (group 1), for a total of 4 times (group 2), and for a total of 7 times (group 3) (A). Also, after tumor was removed from each group, the size and the fluorescence were taken in vitro (B), and then the intensity levels of the fluorescence measured in FIG. 7(B) were converted into numerical values (C); and

FIG. 8 schematically shows the process of in situ amplification of tumor therapeutic effect, and therapeutic response monitoring when a therapeutic agent-containing liposome (e.g., doxorubicin-containing liposome) labeled with an apoptotic cell-targeting peptide and a fluorescent material is used.

### DETAILED DESCRIPTION OF THE DISCLOSURE

Hereafter, the present invention will be described in detail.

The present invention provides use of liposome comprising apoptotic cell-targeting peptides and therapeutic agents and more particularly, the present invention provides a composition for preventing and treating apoptosis-related diseases comprising liposome comprising a peptide having amino acid sequence represented by SEQ ID: No. 1 and an agent for treating apoptosis-related diseases as an active ingredient or a composition for theranosis of apoptosis-related diseases comprising a peptide having amino acid sequence represented by SEQ ID: No. 1, label substances and an agent for treating apoptosis-related diseases as an active ingredient.

Moreover, the present invention provides a method for preventing or treating or theranosis of apoptosis-related diseases comprising the step of administering to a subject in need thereof an effective amount of a peptide having amino acid sequence represented by SEQ ID: No. 1 and an agent for treating apoptosis-related diseases. Also, the present invention provides use of liposome comprising a peptide having amino acid sequence represented by SEQ ID: No. 1 and a therapeutic agent for preparing an agent for treating or theranosis of apoptosis-related diseases.

The apoptosis-related diseases is cancer, myocardial infarction, stroke or arteriosclerosis and an agent for treating means the agent for treating thereof. The peptide of the present invention is capable of specifically binding to apoptotic cells, and thus may be used as an intelligent drug delivery carrier for selectively delivering a drug to the cells. Accordingly, the present invention provides a drug delivery composition comprising the peptide of the present invention as an active ingredient.

In a case where the peptide of the present invention comprised in the drug delivery composition of the present invention is used for treatment in connection with a conventional drug, since the medicine is selectively delivered to only apoptotic cells by the inventive peptide, it is possible to increase the efficacy of the drug, and at the same time to significantly reduce the side effects on a normal tissue.

The peptide of the present invention is an apoptotic cell-targeting peptide (amino acid sequence CQRPPR, ApoPep-1), and is specifically bound to apoptotic cells. The peptide of the present invention (or ApoPep-1 peptide) may have an amino acid sequence of SEQ ID: No. 1 (CQRPPR), and comprise all kinds of peptides, proteins, mimetic peptides, compounds and biomedicines, and have activity capable of specifically binding to apoptotic cells. The peptide of the present invention may be obtained from natural sources, or may be synthesized by using a peptide synthesis method known in the art.

Moreover, the present invention provides a drug delivery method comprising the step of administering to a subject in need thereof an effective amount of a peptide having amino acid sequence represented by SEQ ID: No. 1 and therapeutic agents. Also, the present invention provides use of liposome comprising a peptide having amino acid sequence represented by SEQ ID: No. 1 and an agent for preparing an agent for drug delivery.

As used herein, the “effective amount” refers to the amount effective in drug delivery or preventing or treating apoptosis-related diseases, and the “subject” refers to mammals, particularly, animals comprising human and it may be cells, tissues, organs originated from animals. The subject may be patient in need of treatment.

The peptide of the present invention was selected as which binds specific to apoptotic cells and it binds to the cells by specifically identifying cancer cell in culture status, normal epithelial cell and macrophage. In addition, since the peptide of the present invention targets apoptotic cells within tumor mass, it is possible to perform in vivo imaging and monitoring thereof and delivery of drugs such as anticancer agents, therapeutic agents for myocardial infarction, stroke, arteriosclerosis to each lesion.

Accordingly, the present invention provides a composition for preventing and treating cancer comprising liposome comprising the ApoPep-1 peptide of the present invention and an anticancer agent as an active ingredient and a composition for theranosis of cancer comprising liposome comprising the ApoPep-1 peptide, label substances and an anticancer agent as an active ingredient.

In addition, the present invention provides a method for preventing and treating cancer comprising the step of administering to a subject in need thereof an effective amount of liposome comprising the ApoPep-1 peptide of the present invention and an anticancer agent. Also, the present invention provides use of liposome comprising the ApoPep-1 peptide and an anticancer agent for preparing an agent for preventing and treating cancer.

In addition, the present invention provides a method for theranosis of cancer comprising the step of administering to a subject in need thereof an effective amount of liposome comprising the ApoPep-1 peptide of the present invention, label substances and an anticancer agent. Also, the present invention provides use of liposome comprising the ApoPep-1 peptide, label substances and an anticancer agent for preparing an agent for theranosis of cancer.

In the liposome of the present invention, it is preferable that an ApoPep-1 peptide and a label substance are labeled on the surface, respectively, and a therapeutic agent such as an anticancer agent is encapsulate within the liposome or bound to the surface lipid.

In order to confirm the functions of the liposome of the present invention specifically targeting apoptotic cells, the present inventors, through various experiments, found that the liposome of the present invention has a higher therapeutic effect than a conventional liposome, in administration of an anticancer agent. Also, they verified that the liposome of the present invention targets a tumor region treated with an anticancer agent in a tissue, thereby enabling in vivo imaging and monitoring thereof. Accordingly, it was confirmed that the liposome of the present invention may be utilized for a medicine for diagnosis or treatment monitoring of recognizing a tumor region in a tissue, for a medicine for additional treatment, or for a pharmaceutical composition for prevention and treatment of cancer. Furthermore it was confirmed that the liposome may be used for theranosis that combines treatment with diagnosis.

More specifically, in an example of the present invention, phages specifically binding to macrophages separated from a tumor tissue were screened using a commercially available T7 phage library. As a result, through a total of 3 rounds of screening, phages capable of specifically binding to the cells were screened. Through sequencing, it was confirmed that peptides having the amino acid sequence CQRPPR (SEQ ID: No. 1) were mainly screened out.

In another example of the present invention, the binding specificity of the screened peptides to the cells apoptosis-induced by drug treatment was investigated. As a result, the peptide was strongly bound to the apoptotic cells treated with the drug, whereas it was hardly bound to the drug-untreated cells. Moreover, the binding of the screened peptide to the apoptotic cells was not inhibited by the previous treatment of annexin V at a high concentration. Also, the peptide was confirmed to recognize and bind to the cells in the later stage of apoptosis as well as the early stage.

In another example of the present invention, it was investigated whether a doxorubicin-containing liposome, labeled with an apoptotic cell-targeting peptide, can selectively deliver a drug to a tumor xenotransplanted under the skin of a nude mouse. As a result, the inventive liposome showed a higher therapeutic effect than doxorubicin alone or a doxorubicin-containing liposome itself.

In another example of the present invention, it was investigated whether both selective drug delivery and therapeutic effect can be imaged at once. Herein, a doxorubicin-containing liposome, labeled with an apoptotic cell-targeting peptide together with a near infrared fluorescent material, was injected to a tumor xenotransplanted under the skin of a nude mouse. As a result, as compared to a doxorubicin-containing liposome itself, a liposome labeled with an apoptotic cell-targeting peptide showed a higher therapeutic effect, and a reduced tumor size. At the same time, it was found that through imaging of apoptosis, the peptide-labeled liposome showed a stronger fluorescence signal.

In conclusion, it was confirmed that the liposome of the present invention can specifically deliver a drug to tumor cells in vivo by the inventive peptide. Also, such delivery can be monitored by a label substance. Thus, it can be found that both treatment and diagnosis of cancer can be performed at once.

The liposome of the present invention has a self-assembling structure including one or more lipid bilayers of amphipathic lipid molecules each of which encloses an internal volume. The amphipathic lipid molecules that make up the lipid bilayers include a polar (hydrophilic) head group region covalently linked to one or two non-polar (hydrophobic) acyl chains. The energetically unfavorable contact between the hydrophobic acyl chains and the aqueous media causes the lipid molecules to rearrange, and thus, the polar head groups are oriented towards the aqueous media while the acyl chains are effectively shielded from coming into contact with the aqueous media. This makes it possible to achieve an energetically stable structure.

Preferably, the liposome of the present invention is a multi-lamellar liposome having two or more lipid bilayers. A multi-layered lipid bilayer provides a large number of walls through which internal substances have to be passed so as to leak out of the liposome toward the external environment. Also, multiple lipid bilayers can maintain internal pH of a liposome for a longer time than a single lipid bilayer.

Multi-lamellar liposomes may be produced by various methods (Cullis et al., in: Liposomes, From Biophysics to Therapeutics (M. J. Ostro, ed.), Marcel Dekker, pp. 39-72 (1987)). Bangham's procedure (J. Mol. Biol. 13:238 (1965)) produces “ordinary” multilamellar vesicles(MLVs). This process relates to dissolution of at least one amphipathic lipid in at least one organic solvent. Then, lipids are dried and then are rehydrated by an aqueous solution so as to form MLVs. These ordinary MLVs generally have unequal solute distribution amongst their aqueous compartments. Lenk et al.(U.S. Pat. Nos. 4,522,803, 5,030,453 and 5,169,637), Fountain et al.(U.S. Pat. No. 4,588,578) and Cullis et al.(U.S. Pat. No. 4,975,282) discloses methods for producing multi-lamellar liposomes having an entrapped solute in each of their aqueous compartments.

The multi-lamellar liposome of the present invention generally has a diameter of 5 microns or less, preferably of 1 micron or less. Most preferably, the liposome has a diameter of 50 nm to 500 nm. The size of the liposome may be reduced by various methods known to those skilled in the art. For example, through a filter having holes with a predetermined size, liposomes can be extruded twice or more under pressure (Cullis et al., U.S. Pat. No. 5,008,050; and Loughrey et al.(U.S. Pat. No. 5,059,421). The liposome size may be measured by various methods such as freeze-fracture electron microscopy and quasi-elastic light scattering.

Liposomes can be loaded with bioactive agents by solubilizing the molecules in the medium in which the liposomes are formed, in the case of water-soluble agents, or adding lipid-soluble agents to the lipid solutions from which the liposomes are made. Ionizable bioactive agents can also be loaded into liposomes by establishing an electrochemical potential gradient across the liposomal membrane and then adding the agent to the external medium of the liposomes.

The following literature may be used as a reference for the above-mentioned liposome work (Han et al., Novel cationic cholesterol deriveative-based liposomes for serum-enhanced delivery of siRNA. International Journal of Pharmacuetics, 353;260-269, 2008)

The tumorous disease showing effect on prevention and treatment or theranosis of cancer by the liposome of the present invention are, which are not limited thereto, colon cancer, lung cancer, stomach cancer, esophagus cancer, pancreatic cancer, gallbladder cancer, kidney cancer, bladder cancer, prostate cancer, testicular cancer, cervical cancer, endometrium cancer, choriocarcinoma, ovarian cancer, breast cancer, thyroid cancer, brain cancer, head and neck cancer, malignant melanoma, skin cancer, liver cancer, leukemia, lymphoma, multiple myeloma, chronic myelogenous leukemia, neuroblastoma, or aplitic anemia.

An anticancer agent or anti-tumor agent comprised into the liposome of the present invention may be used without limit as long as they can be used in conventional treatment of cancer or tumor. For example, the existing antitumor medicines are such as paclitaxel, doxorubicin, vincristine, daunorubicin, vinblastine, actinomycin-D, docetaxel, etoposide, teniposide, bisantrene, homoharringtonine, Gleevec; STI-571, cisplain, 5-fluouracil, adriamycin, methotrexate, busulfan, chlorambucil, cyclophosphamide, melphalan, nitrogen mustard), and nitrosourea. The connection between a sample and the lipid of the present embodiment is carried out by a known method such as a simple piling or inclusion and a covalent bonding or cross-linkage. When necessary, the peptide of the present embodiment may be chemically modified without losing its activity.

The liposome may be labeled by well known method to the skilled persons to easily verify and perform a quantitative analysis of the liposome of the present invention at the site of apoptotic cells, especially tumor leisons. Namely, the liposome of the present invention linked to a detectable mark (example: covalent bonding or cross-linkage) may be provided. The detectable mark may be a radioactive isotope (example: ¹²⁵I, ³²P, or ³⁵S), chromophore, a luminescent or a fluorescent material (example: FITC, RITC, Fluorescent Protein (Green Fluorescent Protein (GFP); EGFP(Enhanced Green Fluorescent Protein); RFP(Red Fluorescent Protein); DsRed(Discosoma sp. red fluorescent protein); CFP(Cyan Fluorescent Protein); CGFP(Cyan Green Fluorescent Protein); YFP(Yellow Fluorescent Protein), Cy3, Cy5 and Cy7.5)), super paramagnetic particles, or ultrasuper paramagnetic particles.

Detection techniques based on labeling are widely known in the art. For example, detections may be made as follows. In a case where a fluorescent material is used as a detectable label, immunofluorescence staining may be employed. For example, the inventive liposome labeled with a fluorescent material may be reacted with a test sample, and unbound or unspecifically bound product may be removed. Then, fluorescence emitted by the liposome may be observed under a fluorescent microscope. Also, in a case where an enzyme is used as a detectable label, absorbance may be measured by a color reaction of a substrate through an enzymatic reaction. In a case where a radioactive material is used, a radiation dose may be measured. Furthermore, the detection result may be imaged using a known imaging technique according to the detectable labels.

As described above, apoptosis occurs not only in various kinds of tumor cells, but also in the cells affected by stroke, myocardial infarction or arteriosclerosis (Thomson, Science, 1995, 67:1456-1462; Du et al, J Cereb Blood Flow Metab, 1996, 16:195-201; Narula et al., New Engl J Med, 1996, 335:1182-1189). Accordingly, the drug delivery composition may be specific to cancer or tumoral disease, myocardial infarction, stroke or arteriosclerosis.

Accordingly, the present invention provides a composition for preventing and treating stroke comprising liposome comprising the peptide having amino acid sequence represented by SEQ ID. 1 and a therapeutic agent for stroke as an active ingredient and a composition for theranosis of stroke comprising liposome comprising the peptide having amino acid sequence represented by SEQ ID. 1, label substances and a therapeutic agent for stroke as an active ingredient.

In addition, the present invention provides a method for preventing and treating cancer comprising the step of administering to a subject in need thereof an effective amount of liposome comprising the peptide having amino acid sequence represented by SEQ ID. 1 and a therapeutic agent for stroke. Also, the present invention provides use of liposome comprising the peptide having amino acid sequence represented by SEQ ID. 1 and a therapeutic agent for stroke for preparing an agent for preventing and treating stroke.

In addition, the present invention provides a method for theranosis of stroke comprising the step of administering to a subject in need thereof an effective amount of liposome comprising the peptide having amino acid sequence represented by SEQ ID. 1 and a therapeutic agent for stroke. Also, the present invention provides use of liposome comprising the peptide having amino acid sequence represented by SEQ ID. 1 and a therapeutic agent for stroke for preparing an agent for theranosis of stroke.

The present invention provides a composition for preventing and treating myocardial infarction comprising liposome comprising the peptide having amino acid sequence represented by SEQ ID. 1 and a therapeutic agent for myocardial infarction as an active ingredient and a composition for theranosis of myocardial infarction comprising liposome comprising the peptide having amino acid sequence represented by SEQ ID. 1, label substances and a therapeutic agent for myocardial infarction as an active ingredient.

In addition, the present invention provides a method for preventing and treating myocardial infarction comprising the step of administering to a subject in need thereof an effective amount of liposome comprising the peptide having amino acid sequence represented by SEQ ID. 1 and a therapeutic agent for myocardial infarction. Also, the present invention provides a use of liposome comprising the peptide having amino acid sequence represented by SEQ ID. 1 and a therapeutic agent for myocardial infarction for preparing an agent for preventing and treating myocardial infarction.

In addition, the present invention provides a method for theranosis of myocardial infarction comprising the step of administering to a subject in need thereof an effective amount of liposome comprising the peptide having amino acid sequence represented by SEQ ID. 1 and a therapeutic agent for myocardial infarction. Also, the present invention provides use of liposome comprising the peptide having amino acid sequence represented by SEQ ID. 1 and a therapeutic agent for myocardial infarction for preparing an agent for theranosis of myocardial infarction.

The present invention provides a composition for preventing and treating arteriosclerosis comprising liposome comprising the peptide having amino acid sequence represented by SEQ ID. 1 and a therapeutic agent for arteriosclerosis as an active ingredient and a composition for theranosis of arteriosclerosis comprising liposome comprising the peptide having amino acid sequence represented by SEQ ID. 1, label substances and an therapeutic agent for arteriosclerosis as an active ingredient.

In addition, the present invention provides a method for preventing and treating arteriosclerosis comprising the step of administering to a subject in need thereof an effective amount of liposome comprising the peptide having amino acid sequence represented by SEQ ID. 1 and a therapeutic agent for arteriosclerosis. Also, the present invention provides use of liposome comprising the peptide having amino acid sequence represented by SEQ ID. 1 and a therapeutic agent for arteriosclerosis for preparing an agent for preventing and treating arteriosclerosis.

In addition, the present invention provides a method for theranosis of arteriosclerosis comprising the step of administering to a subject in need thereof an effective amount of liposome comprising the peptide having amino acid sequence represented by SEQ ID. 1 and a therapeutic agent for arteriosclerosis. Also, the present invention provides use of liposome comprising the peptide having amino acid sequence represented by SEQ ID. 1, label substances and a therapeutic agent for theranosis of arteriosclerosis.

In the present invention, a myocardial infarction therapeutic agent or a stroke therapeutic agent may be any one conventionally used for the treatment of the diseases. For example, thrombolytic drugs such as streptokinase, urokinase, alteplase, etc, which are used for removal of thrombus blocking blood vessel in the diseases, may be used. Also, myocardial cell protecting agents such as angiotensin II inhibitor, aldosterone receptor inhibitor, erythropoietin, etc. may be used. Also, brain nerve cell protecting agents such as NMDA (N-methyl-d-aspartate) receptor inhibitor may be used.

Further, there is no limitation in the arteriosclerosis therapeutic agent, as long as it has been conventionally used for the treatment of arteriosclerosis. For example, vascular smooth muscle cell proliferation inhibiting drugs such as Rapamycin, cholesterol synthesis inhibiting or blood cholesterol level reducing drugs such as Lovastatin, anti-inflammatory drugs such as Celebrex, platelet coagulation inhibiting drugs such as Ticlopin, matrix metalloprotease inhibiting drugs such as Marimastat, Trocade, etc. may be used, but the present invention is not limited thereto.

The compositions of the present invention may be a pharmaceutical composition and it may be provided as a formulated form of liposome and/or a pharmacologically permissible carrier. ‘Pharmacologically acceptable’ means a non-toxic composition which does not produce an allergic or a similar reaction such as a stomach disorder or vertigo when the composition is physiologically permissible and medicated to human. The carrier is all kinds of solvent, a dispersion medium, an o/w or w/o emulsion, an aqueous composition, liposome, a microbead, a microsome and biodegradable nanoparticle. Preferably, the pharmaceutical compositions of the present invention may comprise 0.001-99.999 wight % of pharmacologically acceptable carriers.

Also, the composition of the present invention may comprise 0.00001%˜20 wight % of the peptide having the amino acid sequence represented by SEQ ID: No. 1, for example, 0.00001%˜20 wight % of the agent for cancer, myocardial infarction, stroke or arteriosclerosis, 0.01%˜30 wight % of liposome formation ingredient and 30˜99.99 weight %, that is the remaining, of the pharmacologically acceptable carrier.

Meanwhile, the pharmacological compositions may be formulated with a proper carrier according to a medication route. The medication route according to the present invention is an oral or parenteral route, but not limited thereto. The parenteral medication route contains a transdermal, a nasal cavity, an abdominal cavity, a muscle, a hyperdomic, or a vein.

In case of the oral administration of the pharmacological composition of the present invention, it can be formulated in the form of powder, granule, tablets, pills, sugar coated tablets, capsules, fluids, gels, syrups, suspensions wafers, and the like. Example of a proper carrier may comprise a series of saccharide such as lactose, dextrose, sucrose, sorbitol, mannitol, xylitol, erythritol, and maltitol; a series of starch such as corn starch, wheat starch, rice starch, and potato starch; a series of cellulose such as cellulose, methyl cellulose, sodium carboxy methyl cellulose, and hydroxylpropylmethyl cellulose; and a series of filler such as gelatin and polyvinyl pyrrolidone. In some cases, a disintegrants such as cross-linked polyvinyl pyrrolidone, agar, alginic acid, or sodium alginate may be added. The pharmacological compositions may additionally contain a flocculant, a lubricant, a wetting agent, a perfume, an emulsifying agent, or a preservative.

When the pharmacological compositions are used for parenteral administration, the pharmacological composition may be formulated by a known method in the form of an injections, transdermal preparations, and nasal preparation with a proper carrier. The injections have to be sterilized and prevented from microorganism contaminations such as bacteria or fungus. In the case of a injections, the proper carrier is, but not limited thereto, water, ethanol, polyol (example; glycerol, propylene glycol, liquid polyethylene glycol), or a mixture of the above materials and/or a solvent or a dispersion medium containing a vegetable oil. More preferably, the proper carrier is hanks solution, linger solution, phosphate buffered saline containing triethanol amine, a sterilized solution for a injections, or a isotonic solution such as 10% ethanol, 40% propylene glycol, or 5% dextrose. Antimicrobial or antifungal such as paraben, chloro butanol, phenol, sorbic acid, and thimerosal may be added for the prevention of the injections from microorganism contaminations. And, the most of injections may contain an isotonic agent such as sugar or sodium chloride. Those formulations are described in an existing formula known to the pharmaceutical chemistry (Remington's Pharmaceutical Science, 15th Edition, 1975, Mack Publishing Company, Easton, Pa.).

In the case of a nasal preparation, the compounds used in the present invention are easily delivered in the form of aerosol spray from a pressurized pack or a nebulizer. For the production of a nasal preparation, the proper propellant such as dichlorofluoro methane, trichlorofluoro methane, dichlorotetrafluoro ethane, carbon dioxide, or the other proper gas is used. In the case of pressurized aerosol, a dosage unit is determined by a valve delivering a measured quantity. For instance, a gelatin capsule or a cartridge used in an inhaler or an insufflator may be formulated to contain a powder base such as lactose or starch.

The other pharmaceutically acceptable carriers may be referred from the below-mentioned literature (Remington's Pharmaceutical Sciences, 19th ed., Mack Publishing Company, Easton, Pa., 1995).

Furthermore, the pharmacological compositions according to the present invention may additionally contain one or more buffer (example; NaCl solution or PBS), a carbohydrate (example; glucose, mannose, sucrose, or dextran), a stabilizer (example; sodium bisulfate, sodium sulfite, or ascorbic acid), an antioxidant, a bacteriostat, a chelating agent (example; EDTA or glutathione), an adjuvant (example; aluminum hydroxide), a suspension agent, a thickener, and/or a preservative (example; benzalkonium chloride, methyl or propylparaben, or chlorobutanol).

Also, the inventive pharmaceutical composition may be formulated by using the method known in the art so that it can provide rapid, continuous or delayed release of an active ingredient after it is administered to a mammalian.

An effective amount of the pharmacological compositions formulated by the above methods are administered to a number of routes such as oral, transdermal, hypodermic, vein, or muscle. Here, ‘effective amount’ means an amount of a compound or an extract which makes it possible to trace a treatment effect or diagnosis when the pharmacological composition is medicated to a patient. A dosage of the pharmacological composition according to the present invention is selected by a administration route, a administering subject, a type and a degree of a serious illness of a disease, an age, sex and body weight, individual differences, and a disease condition. Preferably, the content of an active ingredient in the pharmacological composition of the present invention may be varied by a disease condition, and I may be administered with an effective amount in a dosage of several times a day.

Moreover, the liposome of the present invention is specifically bound to apoptotic cells, and thus it may be useful for imaging and diagnosis of the lesion of cancers, timorous diseases, stroke, myocardial infarction, or arteriosclerosis. At this time, imaging and diagnosis of diseases are, not limited thereto, used for monitoring of the progress of diseases, the result of treatment, reaction against therapeutic agent as well as the first medical examination. Accordingly, the composition of the present invention may be used for theranosis.

As can be seen foregoing, the liposome comprising the peptide of the present invention targets specifically to apoptotic cells and able to deliver therapeutic agent comprised in the liposome. Accordingly, the present invention may be used for drug delivery to the apoptotic cells in cancer or tumor mass, the apoptotic myocardial cells in myocardial infarction lesion, the apoptotic stroke cells in stroke lesion, the apoptotic cells in arteriosclerosis lesion and further may be used for detection of the cells and imaging diagnosis. Therefore, it can be used for theranosis as well as preventing or treating the diseases.

### EXAMPLES

Hereinafter, the present invention will be described in detail with reference to Examples.

However, Examples below are for illustrative purpose only and are not constructed to limit the scope of the present invention.

### Example 1

### Screening of Peptide Having Binding Specificity to Apoptotic Cells

<1-1> Preparation of Phage Peptide Library

In order to find out peptides specific to apoptotic cells from among various cells constituting a tumor tissue, the present inventors employed the phage peptide display technique (Smith, Science, 228:1315-1317, 1985). Phage peptide display refers to displaying peptides composed of several to several tens of amino acids on the surface of bacteriophage. Since a phage library with as many as 10⁹ peptides can be prepared, the technique is useful in screening a large number of peptides at once and finding out the peptides targeting a desired tissue or cell.

The phage peptide library used in the present invention was prepared as follows. Oligonucleotides coding CX₇C peptides having cysteine at both ends and 7 random amino acids between them were randomly synthesized. The synthesis of the oligonucleotides was carried out by Macrogen (Korea). Then, the synthesized oligonucleotides were cloned into the capsid protein gene constituting the surface of T7 415-1b phage by using a T7Select phage cloning kit of Novagen (USA) according to the manufacturer's instructions, thereby preparing phage peptide library. The diversity of the prepared phage peptide library was measured at about 5×10⁸ pfu.

<1-2> Screening of Phage Peptide Library

Tumor tissues and normal tissues neighboring the tumors, which had been obtained from surgical operations for tumor treatment, were finely cut using a knife, and grinded using a tissue homogenizer to prepare a cell suspension. The phage library prepared in Example <1-1> was mixed with the cell suspension obtained from the normal tissue, and they were allowed to react at 4° C. for hours. After the reaction was completed, only the supernatant was taken. After the phages not bound to normal cells were collected, and the titer was amplified using BL21 E. coli as host. Subsequently, the cell suspension obtained from the tumor tissue was reacted under the same condition. The phages non-specifically and weakly binding to tumor cells were removed by washing with 1 ml of a DMEM solution (Dulbeco's modified Eagle's medium) containing 1% bovine serum albumin (BSA) for 5 minutes at room temperature, for a total of 3 times. Following the washing, magnetic beads on which anti-macrophage antibody (anti-CD14 antibody, Dynal) or anti-endothelial cell antibody (anti-CD31 antibody, Dynal) was attached were reacted with the cell suspension at 4° C. for 30 minutes. Then, the cells adhering to the respective magnetic beads were isolated by a magnet. The isolated macrophages or endothelial cells were treated with 100 μl of DMEM solution containing 1% NP-40 at 4° C. for 10 minutes. Then, after adding 900 μl of BL21 E. coli culture medium as a host, the phages binding to the cells were detected. The titer was measured for a part of the detected phages according to a method known in the art (Phage display, Clackson T and Lowman H B, p. 171, 2004, Oxford University Press, New York). The remaining phages were amplified. Then, the procedure of the screening of phages binding to respective cells was repeated for a total of 3 times in the same manner as described above. As a result, the titer of the phages binding to the macrophages and endothelial cells derived from the tumor tissue sequentially significantly increased. Thus, it was found that the screening was successfully performed (data not shown).

<1-3> DNA Sequencing and Amino Acid Sequencing of Phage Clone

In order to investigate which peptide was displayed for the phages screened in Example <1-2>, 30 phage clones were randomly selected for each cell, and the DNA inserted in the phages was amplified by PCR and sequenced. Herein, the 5′-primer was the oligonucleotide (AGCGGACCAGATTATCGCTA, sequence ID NO: 2) and the 3′-primer was the oligonucleotide (AACCCCTCAAGACCCGTTTA, sequence ID No 3). PCR was carried out with pre-denaturation of template DNA for 5 minutes at 95° C., 35 cycles of 50 seconds at 94° C.; 1 minute at 50° C.; and 1 minute at 72° C., and final extension for 6 minutes at 72° C.

The PCR product was sequenced by DNA sequencing company (Bioneer). Based on the resultant base sequence, the amino acid sequence was deduced. Through analysis of the deduced amino acid sequence using the ClustalW program, the peptides of the representative phage clones most frequently occurring for the macrophages and endothelial cells were obtained, respectively. They represented sequence ID No 1(ApoPep-1, CQRPPR, screened for the macrophages).

### Example 2

### Binding of the Inventive Peptide to Apoptotic Cells

<2-1> Microscopic Observation of Binding of the Peptide to Apoptotic Cells

Cells were cultured in a chamber slide (Nalgen Nunc), and treated with etoposide (Sigma) at a concentration of 50 μM for a given period of time to induce apoptosis (A549 and HeLa cells: for 15 hours, H460 cells: 24 hours, L132 cells: 3 hours, and RAW cells: 6 hours). The cells were cultured in RMPI-1640 medium (A549 and H460 cells) or DMEM medium (HeLa, L132 and RAW cells) containing antibiotics (penicillin and streptomycin) and 10% FBS. Meanwhile, all the cells were subcultured every 3 or 4 days. The apoptosis-induced apoptotic cells were washed with PBS, and blocked with 1% BSA at 37° C. for 30 minutes. Then, the cells were reacted with 10 μM of the peptide labeled with fluorescein, at 4° C. for 1 hour. After being washed, the cells were reacted with an annexin V reaction buffer solution containing alexa 594 (fluorescent reagent)-labeled annexin V (Molecular Probes) at room temperature for 15 minutes. The cells were washed with PBS, and then fixed with 4% paraformaldehyde for 5 minutes. Then, after counterstaining using the nuclear stain 4′,6-diamidino-2-phenylindole (DAPI), followed by treatment with a mounting solution (Molecular Probes), images of the cells were obtained under a fluorescence microscope (Zeiss).

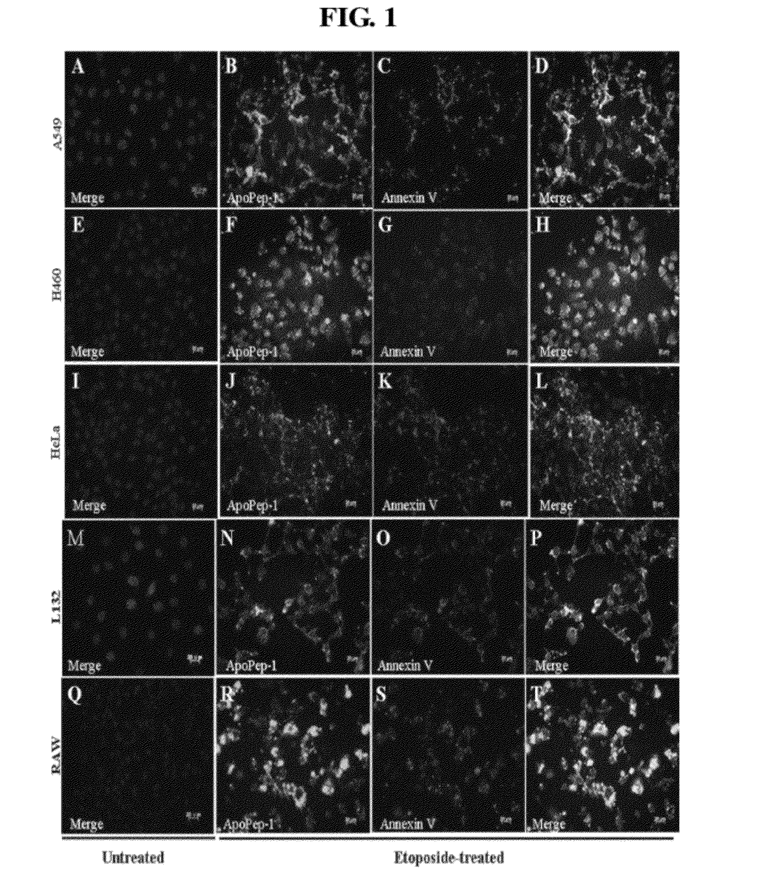

As a result, as shown in FIG. 1, no labeling was observed when the normal cells, not treated with etoposide, were treated with the inventive peptide (ApoPep-1) and annexin V (first column in FIG. 1: A, E, I, M, O). In contrast, labeling was observed when etoposide-treated apoptotic cells were treated with the inventive peptide (ApoPep-1) (second column in FIG. 1: B, F, J, N, R) or with annexin V (third column in FIG. 1: C, G, K, O, S). Through merging of the images using a computer program, it was confirmed that the bindings for both the inventive peptide and annexin V were at the same locations (fourth column in FIG. 1: D, H, L, P, T).

<2-2> Competitive Inhibition of Binding of the Peptide to Apoptotic Cells by Treatment of Annexin V

In order to further investigate the binding properties of the inventive peptide (ApoPep-1) to apoptotic cells, competitive inhibition by annexin V was measured. For this, first, apoptotic A549 cells were pretreated with annexin V, not labeled with fluorescence, at concentrations of 0, 200 and 1000 μM. Then, after the cells were reacted with fluorescence-labeled annexin V under the same condition as described in Example <2-1>, the binding of the cells was observed under a fluorescence microscope.

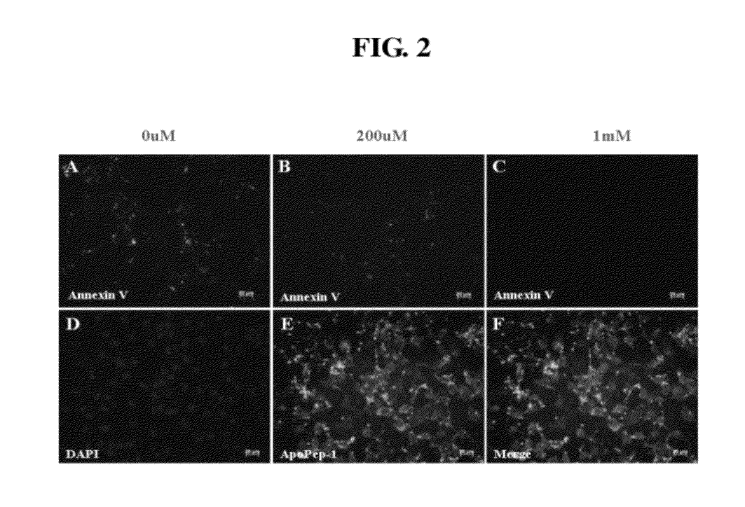

As a result, as shown in FIGS. 2A to 2C, the fluorescence significantly decreased when annexin V, not labeled with fluorescence, was pre-treated at high concentration, due to competitive inhibition of the binding with fluorescence-labeled annexin V.

Meanwhile, apoptotic A549 cells were pre-treated with annexin V, not labeled with fluorescence, at a concentration of 1000 μM. Then, after the cells were reacted with fluorescence-labeled peptide under the same condition as described in Example <2-1>, the binding of the cells was observed under a fluorescence microscope.

As a result, as shown in FIGS. 2D to 2F, the binding of the inventive peptide(ApoPep-1) was not inhibited by the pre-treatment with annexin V at a high concentration.

<2-3> Confirmation of Binding of the Inventive Peptide to Apoptotic Cells Through FACS Analysis

As another way of confirming the binding of the inventive peptide to apoptotic cells, apoptotic cells were treated with the inventive peptide labeled with fluorescein, and the binding was confirmed through FACS analysis. First, apoptosis was induced by treating A549 cells with 50 μM etoposide for 6 to 15 hours. The apoptotic cells or normal cells were reacted with the inventive ApoPep-1 peptide(5 μM) or a control peptide at the same concentration, labeled with fluorescein, at 4° C. for 1 hour. Meanwhile, the cells were reacted with fluorescein-labeled annexin V at room temperature for 15 minutes. After simultaneously staining the cells with propodium iodide (PI), followed by washing with PBS, FACS analysis was performed using a FACS instrument (Becton Dickinson).

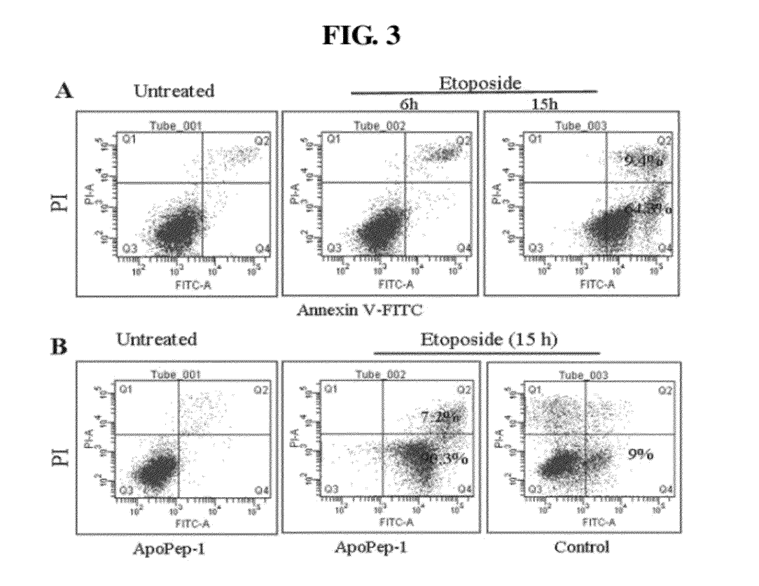

As a result, as shown in FIG. 3, when the etoposide-treated apoptotic A549 cells were stained with annexin V and PI, the percentage of the cells stained only by annexin V (fraction Q4, early stage of apoptosis) and the percentage of the cells stained by both annexin V and PI (fraction Q2, later stage of apoptosis) were 64.3% and 9.4%, respectively, at 15 hours, which were higher than at 6 hours (see FIG. 3A). Meanwhile, when the cells that had been treated with etoposide for 15 hours were treated with the inventive ApoPep-1 peptide, 90.3% and 7.2% of the cells at the early stage and later stage of apoptosis, respectively, were bound to the peptide (see FIG. 3B). On the other hand, when the apoptotic cells were treated with the control peptide or when the normal cells were treated with the inventive peptide (ApoPep-1), the binding of cells was almost nonexistent.

### Example 3

### Preparation of Doxorubicin-Containing Liposome Labeled with Apoptosis-Targeting Peptide and Near Infrared Fluorescent Reagent

<3-1> Preparation of Doxorubicin-Containing Liposome Labeled with Peptide and Near Infrared Fluorescent Reagent

Reagents such as egg L-α-phosphatidylcholine (PC), egg L-α-phosphatidyl-DL-glycerol (PG), 1,2-diacyl-sn-glycero-3-phosphoethanolamine-N-[methoxy(polyethylene glycol)-2000]; mPEG₂₀₀₀-DSPE), (1,2-distearoyl-sn-glycero-3-phosphoethanolamine-N-[maleimide(polyethyleneglycol) 2000]; maleimide-PEG₂₀₀₀-DSPE), 1,2-dioleoyl-sn-glycero-3-phosphoethanolamine (DOPE) were bought from Avanti Polar Lipids. Apoptosis-targeting peptide (15 μmol) was dissolved in water at room temperature, and added with DTT (dithithreitol) to a concentration of 1 mM, followed by stirring for 1 hour. The resultant solution was added with maleimide-PEG₂₀₀₀-DSPE dissolved in dimethylsulfoxide, followed by stirring at 4° C. for 16 hours. From the resultant solution, a solvent was removed by using a rotary evaporator, and then ethyl acetate was added thereto. Through purification using dialysis, colorless and viscous ApoPep-1-PEG₂₀₀₀-DSPE was obtained.

Cy™ 7.5 hydroxysuccinimide (15) was dissolved in water, and added to dimethylformamide having DOPE dissolved therein at room temperature. PG was used for obtaining negative charges on the liposome surface. The resultant mixture was stirred for 16 hours, and evaporated to completely remove the solvent. Then, the mixture was washed with ethyl acetate. Then, the mixture was purified with column chromatography so as to finally provide green and viscous Cy™ 7.5-DOPE.

Liposomes were prepared by a multi-lamellar vesicle method. First, PC, PG, cholesterol, Cy7.5-DOPE and ApoPep-1-PEG₂₀₀₀-DSPE were dissolved in chloroform with a molar ratio of 5:5:5:0.1:0.2. PG was used for giving negative charges on the liposome surface. The lipid mixture was placed within a decompressed rotary evaporator so as to remove chloroform. Then, thin lipid films were formed. The formed lipid films were hydrated with 20 mM HEPES buffer (pH 7.4) aqueous solution, and then vortexed to form multi-lamellar vesicles. Then, by repeatedly passing them through polycarbonate membrane filters (hole size: 200 nm), the liposome size was homogenized. The liposome solution was added with 500 g of doxorubicin with respect to 1 mL of the liposome solution, followed by stirring (PC:PG:Cholesterol:Cy7.5-DOPE:ApoPep-1-PEG-DSPE=5:5:5:0.1:0.2 mole/mL). Doxorubicin has an amine group at neutral pH (7.4), and thus is positively charged. Thus, it is electrostatically bound to negatively-charged PG-containing liposomes. Remaining unbounded doxorubicin was removed by gel filtration through Sephadex™ G-25M column (GE Healthcare).

### Example 4

Selective Drug Delivery and Tumor-Target Therapy Using Doxorubicin-Containing Liposome Labeled with Apoptosis-Targeting Peptide

<4-1> Preparation of Tumor-Xenotransplanted Model Nude Mouse

All animal experiments were performed in accordance with the guidelines of the institutional animal care and use committee. For tumor xenografts, human lung cancer cell lines (H460 and A549, 1×10⁷ cells) suspended in RMPI-1640 medium were subcutaneously injected at the right upper or lower limb of a 6 week old male BALB/c nude mouse (Hyochang Science). Then, 3 weeks were given for the tumor cells to grow to a size of 0.5 to 1 cm. The H460 cell line used in this experiment was cultured in RMPI-1640 medium containing 10% FBS (Fetal bovine serum) added with antibiotics (penicillin and streptomycin). Subculturing was performed every 3 or 4 days.

<4-2> Selective Drug Delivery and Target Therapy of Doxorubicin-Containing Liposome by the Medium of Peptide

In order to determine the drug delivery facilitating effect by the medium of an apoptosis-targeting peptide, a liposome which contains doxorubicin (currently used for anticancer treatment) and/or is coated with polyethylene glycol was prepared in the same manner as described in Example <3-1>, and the surface of the liposome was labeled with an ApoPep-1 peptide. A tumor-xenotransplanted nude mouse was prepared by using H460 lung cancer cell lines in the same manner as described in Example <4-1>. When the tumor was grown to a diameter of about 3 mm, the liposome solution was intravenously injected in such a manner that the doxorubicin is injected at 1 mg/kg body weight of a mouse. The liposome solution was injected for a total of seven times with a 2 day interval. For 44 days, the tumor size was measured with a 2 day interval, and the body weight of the mouse was measured with a 4 day interval.

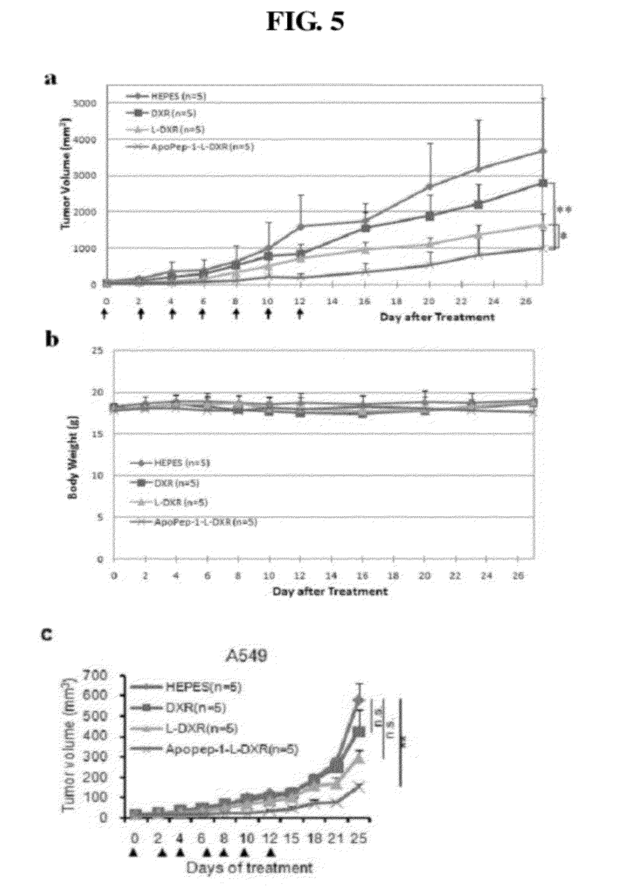

As a result, the administration of the liposome labeled with the apoptosis-targeting peptide showed a higher H460 tumor growth inhibiting effect than the administration of a non-labeled liposome or doxorubicin itself at the same concentration (see FIG. 5A). Meanwhile, in view of body weight, respective groups showed no significant difference (see FIG. 5B).

Also, in a case of A549 tumor, the administration of the liposome labeled with the apoptosis-targeting peptide showed a higher tumor growth inhibiting effect than the administration of a non-labeled liposome or doxorubicin itself at the same concentration (see FIG. 5C).

### Example 5

Selective Drug Delivery and Theranosis Using Doxorubicin-Containing Liposome Labeled with Apoptosis-Targeting Peptide and Near Infrared Fluorescent Reagent

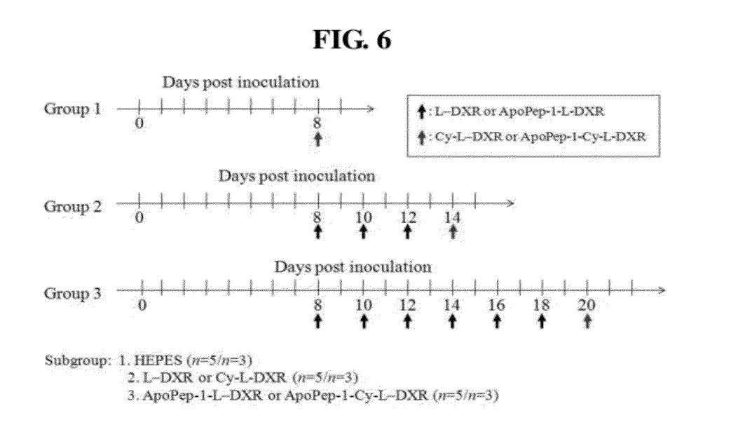

Theranosis is a compound word of therapy with diagnosis (diagnostics or imaging), which indicates simultaneous performance of both a therapy technique and a diagnosis technique (through imaging, etc.). For this, a doxorubicin-containing liposome (ApoPep-1-L-DXR), labeled with an apoptosis-targeting peptide (ApoPep-1), and a doxorubicin-containing liposome (ApoPep-1-Cy-L-DXR), labeled with both the peptide and a Cy7.5 near infrared fluorescent reagent, were prepared. Tumor-xenotransplanted nude mice were divided into three groups consisting of three mice in each group. On day 8 from xenotransplantation, ApoPep-1-L-DXR was firstly intravenously injected (group 1), and then injected with a 2 day interval for a total of 4 times (group 2) and for a total of 7 times (group 3) (see FIG. 6). For each group, near infrared fluorescent reagent-labeled ApoPep-1-Cy-L-DXR was lastly injected (see FIG. 6). After 2 hours from the injection, mice were put under anesthesia while in vivo images on near infrared fluorescence at tumor regions were obtained.

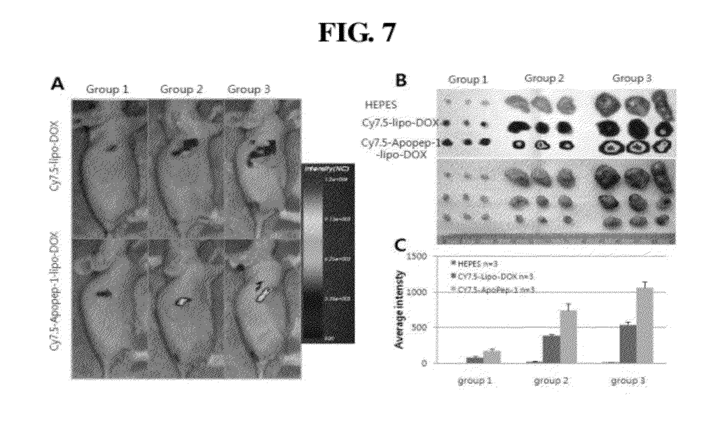

As a result, as compared to groups 1 and 2, group 3 showed a stronger near infrared fluorescence level in the tumor according to the increase of the number of times of liposome injections. Especially, the apoptosis-targeting peptide-labeled liposome (ApoPep-1-Cy-L-DXR) showed a much stronger fluorescence signal than the peptide-non-labeled liposome (Cy-L-DXR) (see FIG. 7A). Also, from each group, tumor was removed and its size and fluorescence level were in vitro photographed. As a result, it was found that the group treated with the apoptosis-targeting peptide-labeled liposome showed a reduced tumor size but a stronger fluorescence signal, compared to a control group (see FIG. 7B). FIG. 7C shows the average and standard deviation of the numerical values obtained through conversion of the intensity levels of the fluorescence measured in FIG. 7B.

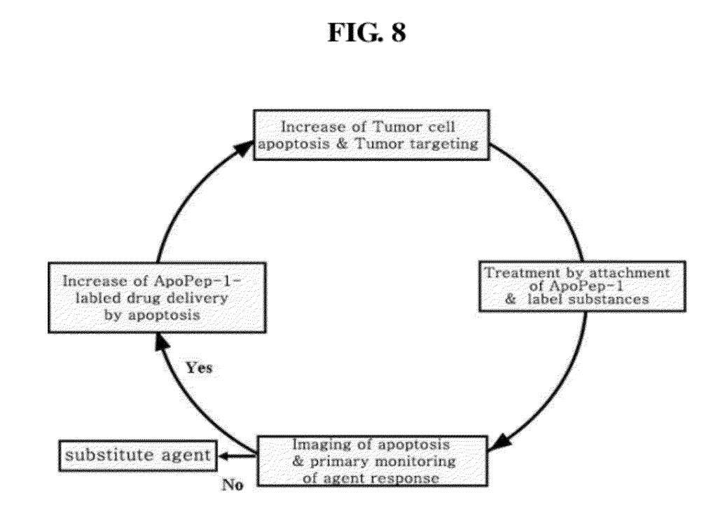

FIG. 8 schematically shows the process of in situ amplification of tumor therapeutic effect, and therapeutic response monitoring when a therapeutic agent-containing liposome (e.g., doxorubicin-containing liposome) labeled with the apoptotic cell-targeting peptide and the fluorescent material is used, as described in Examples above. In other words, when apoptosis of tumor cells is induced by the therapeutic agent, this apoptosis is confirmed through binding with the labeling material. Through continuous treatment, due to the characteristic of the apoptotic cell-targeting peptide, a larger amount of therapeutic agents can be transferred to the tumor tissue. As a result, this may induce apoptosis of more tumor cells, thereby gradually amplifying tumor targeting. This may be called in situ amplification of tumor targeting. On the other hand, when apoptosis is not induced, such a state can be quickly determined through images. This may be helpful in determining to replace the therapeutic agent with another agent.

As can be seen foregoing, the liposome comprising the peptide of the present invention targets specifically to apoptotic cells and able to deliver therapeutic agent comprised in the liposome. Accordingly, the present invention may be used for drug delivery to the apoptotic cells in cancer or tumor mass, the apoptotic myocardial cells in myocardial infarction lesion, the apoptotic stroke cells in stroke lesion, the apoptotic cells in arteriosclerosis lesion and further may be used for detection of the cells and imaging diagnosis. Therefore, it can be used for theranosis as well as preventing or treating the diseases.

1. A drug delivery composition comprising liposome comprising a peptide having the amino acid sequence represented by SEQ ID: No. 1 and therapeutic agents as an active ingredient.

2. A composition for preventing and treating cancer comprising liposome comprising the peptide of claim 1 and an anticancer agent as an active ingredient.

3. The composition of claim 2, wherein the cancer is selected from the group consisting of colon cancer, lung cancer, stomach cancer, esophagus cancer, pancreatic cancer, gallbladder cancer, kidney cancer, bladder cancer, prostate cancer, testicular cancer, cervical cancer, endometrium cancer, choriocarcinoma, ovarian cancer, breast cancer, thyroid cancer, brain cancer, head and neck cancer, malignant melanoma, skin cancer, liver cancer, leukemia, lymphoma, multiple myeloma, chronic myelogenous leukemia, neuroblastoma, or aplitic anemia.

4. The composition of claim 2, wherein the anticancer agent is selected from the group consisting of paclitaxel, doxorubicin, vincristine, daunorubicin, vinblastine, actinomycin-D, docetaxel, etoposide, teniposide, bisantrene, homoharringtonine, Gleevec; STI-571, cisplain, 5-fluouracil, adriamycin, methotrexate, busulfan, chlorambucil, cyclophosphamide, melphalan, nitrogen mustard), and nitrosourea.

5. A composition for theranosis of cancer comprising liposome comprising the peptide of claim 1, label substances and an anticancer agent as an active ingredient.

6. The composition of claim 5, wherein the label substances are selected from the group consisting of a chromogenic enzyme, a radioactive isotope, chromophore, a luminescent, or a fluorescer, super paramagnetic particles, or ultrasuper paramagnetic particles.

7. A composition for preventing and treating stroke comprising liposome comprising the peptide of claim 1 and a therapeutic agent for stroke as an active ingredient.

8. A composition for theranosis of stroke comprising liposome comprising the peptide of claim 1, label substances and a therapeutic agent for stroke as an active ingredient.

9. A composition for preventing and treating myocardial infarction comprising liposome comprising the peptide of claim 1 and a therapeutic agent for myocardial infarction as an active ingredient.

10. A composition for theranosis of myocardial infarction comprising liposome comprising the peptide of claim 1, label substances and a therapeutic agent for myocardial infarction as an active ingredient.

11. A composition for preventing and treating arteriosclerosis comprising liposome comprising the peptide of claim 1 and a therapeutic agent for arteriosclerosis as an active ingredient.

12. A composition for theranosis of arteriosclerosis comprising liposome comprising the peptide of claim 1, label substances and a therapeutic agent for arteriosclerosis as an active ingredient.

13. A drug delivery method comprising the step of administering to a subject in need thereof an effective amount of liposome comprising the peptide of claim 1 and a therapeutic agent.

14. Use of liposome the peptide of claim 1 and a therapeutic agent for preparing an agent for drug delivery.

15. A method for preventing and treating cancer comprising the step of administering to a subject in need thereof an effective amount of liposome comprising the peptide of claim 1 and an anticancer agent.

16. Use of liposome the peptide of claim 1 and an anticancer agent for preparing an agent for preventing and treating cancer.

17. A method for theranosis of cancer comprising the step of administering to a subject in need thereof an effective amount of liposome comprising the peptide of claim 1, label substances and an anticancer agent.

18. Use of liposome comprising the peptide of claim 1, label substances and an anticancer agent for preparing an agent for theranosis of cancer.

19. A method for preventing and treating stroke comprising the step of administering to a subject in need thereof an effective amount of liposome comprising the peptide of claim 1 and a therapeutic agent for stroke.

20. Use of liposome comprising the peptide of claim 1 and a therapeutic agent for stroke for preparing an agent for preventing and treating stroke.

21. A method for theranosis of stroke comprising the step of administering to a subject in need thereof an effective amount of liposome comprising the peptide of claim 1, label substances and a therapeutic agent for stroke.

22. Use of liposome comprising the peptide of claim 1, label substances and a therapeutic agent for stroke for preparing an agent for theranosis of stroke.

23. A method for preventing and treating myocardial infarction comprising the step of administering to a subject in need thereof an effective amount of liposome comprising the peptide of claim 1 and a therapeutic agent for myocardial infarction.

24. Use of liposome comprising the peptide of claim 1 and a therapeutic agent for myocardial infarction for preparing an agent for preventing and treating myocardial infarction.

25. A method for theranosis of myocardial infarction comprising the step of administering to a subject in need thereof an effective amount of liposome comprising the peptide of claim 1, label substances and a therapeutic agent for myocardial infarction.

26. Use of liposome comprising the peptide of claim 1, label substances and a therapeutic agent for myocardial infarction for preparing an agent for theranosis of myocardial infarction.

27. A method for preventing and treating arteriosclerosis comprising the step of administering to a subject in need thereof an effective amount of liposome comprising the peptide of claim 1 and a therapeutic agent for arteriosclerosis.

28. Use of liposome comprising the peptide of claim 1 and a therapeutic agent for arteriosclerosis for preparing an agent for preventing and treating arteriosclerosis.

29. A method for theranosis of arteriosclerosis comprising the step of administering to a subject in need thereof an effective amount of liposome comprising the peptide of claim 1, label substances and a therapeutic agent for arteriosclerosis.

30. Use of liposome comprising the peptide of claim 1, label substances and a therapeutic agent for arteriosclerosis for preparing an agent for theranosis of arteriosclerosis.
